(19) United States
(12) Patent Application Publication
(10) Pub. No.: US 2005/0277113 A1
(43) Pub. Date: Dec. 15, 2005

(54) Saliva immunoassay for detection of exposure to infectious agents

(76) Inventor: Aristo Vojdani, Los Angeles, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/031,864

(22) Filed: Jan. 7, 2005

Related U.S. Application Data
(62) Division of application No. 10/007,768, filed on Nov. 8, 2001, now abandoned.

(51) Int. Cl. C12Q 1/70; G01N 33/554; G01N 33/569; G01N 33/53; G01N 33/537; G01N 33/543

(52) U.S. Cl. 435/5; 435/7.32; 435/7.92

(57) ABSTRACT

A method for diagnosing the exposure to infectious agents in a patient is disclosed. The method determines the levels of antibodies against infectious agents, including bacterial, parasitic, and viral agents. It then compares the results to normal levels to determine the exposure to infectious agents.

### RELATED APPLICATION

This application is a divisional application of U.S. application Ser. No. 10/007,768, filed Nov. 8, 2001.

### BACKGROUND OF THE INVENTION

1. Field of the Invention

The invention relates to a saliva immunoassay for detection of exposure to infectious agents.

2. Description of the Related Art

Pathogenesis of cardiovascular autoimmune disease and other autoimmune diseases induced by infectious agents is described by three different mechanisms of action: release of toxins or superantigens, induction of inflammation, and molecular mimicry or cross-reactivity. This may result in plaque formation or antimyosin cellular and humoral immunity and subsequently, to myocarditis or other autoimmune diseases.

Through the years, many reports have incriminated various infectious agents in the pathogenesis of autoimmune disease. Moreover, the American College of Cardiology has issued a list of harmful pathogens as possible links to heart disease.

Traditionally, it is assumed that infectious agents induce disease by direct tissue damage via secretion of toxins or different antigens, particularly myosin. These toxins may directly or indirectly induce tissue damage and cause release of tissue antigens.

An infectious agent can be taken up by macrophages and transferred to the bloodstream and arteries. When a macrophage burrows into the wall of a blood vessel to take in irritants such as LDL and oxidized LDL, it transfers the infectious agent into the neighboring arterial cells. Infected arterial cells then attract more macrophages and other inflammatory responses, such as platelets, and then die. If this vicious cycle of inflammation continues, it can result in fibrous lesions or plaque formation. When pieces of the plaque break loose, they can start blood clots and cause heart attack.

Another mechanism by which infectious agents can cause autoimmune disease is molecular mimicry. Molecular mimicry is defined as structural similarity between antigens coded by different genes. Antigenic cross-reactivity between host and bacteria is exemplified by blood group substances and bacterial polysaccharides; cardiac tissue and streptococcal proteins; and kidney tissue and E. coli polysaccharides. Viruses may also induce autoimmune responses through shared determinants on molecules notably present on host cells, by altering the host immune system, or by causing the expression or release of “normally sequestered” self antigens.

Harmful pathogens may be the cause of many human diseases. These pathogens may induce their pathologic response through one of the above-mentioned mechanisms of action.

Many viruses, bacteria, and even parasites are claimed to affect atherosclerosis plaque deposition. Among them, Chlamydia pneumoniae probably has the strongest association with atherosclerosis. There is a close relationship between C. pneumoniae infection, IgG and IgM titers, and increased evidence of MI, CVA, and peripheral vascular disease (PVD). C. pneumoniae antigens are found in atherosclerosis plaques, and T-cell reactions to these antigens have been demonstrated. Experimental models illustrate the pathogenic role of C. pneumoniae and the unique heat shock protein (HSP)-60. Other major atherosclerosis-associated pathogens are Helicobacter pylori, Epstein-Barr virus and cytomegalovirus. For some pathogens, interfering pathogenic mechanisms have been described, such as cytomegalovirus gene-induced proliferation of smooth-muscle cells. From data showing a correlation between increased atherosclerosis incidence and chronic bronchitis, as well as periodontitis, it has been suggested that any infectious agent, and especially multiple chronic infections, could result in accelerated atherosclerosis formation. This multiplicity was confirmed recently in experimental animal models. There is no doubt therefore, that chronic infections with specific or nonspecific infectious agents can contribute to the acceleration of atherosclerosis development, either by nonspecific mechanisms [hypercoagulation and increased adhesion molecule and elevated C-reactive protein (CRP) levels] or by more specific mechanisms, such as induction of HSP-60 expression and eventually pathogenic anti-HSP-60 antibody production.

For years it has been known that Chlamydia can induce cardiovascular disease in experimental animals. This Chlamydia-mediated heart disease in mice can be induced by antigenic mimicry of a heart muscle-specific protein, thus providing a molecular link between Chlamydia infections and heart disease. Since many infectious agents have been implicated in heart disease, it is not surprising that organisms other than Chlamydia can also supply mimicking epitopes. Indeed, Machmaier, K. et al., in a study published in Nature Medicine in August 2000, screened public databases for proteins sharing the pathogenic mouse M7Aαpeptide MA'ST motif (whose amino acid sequence is as follows: SLKLMATLFSTYASA). This motif is found in proteins from a multitude of viruses, bacteria, fungi, and protozoa, which are involved in cardiovascular disease.

Manifestation of Antibodies

The deposition of antigens in the gut has been shown to lead to the production of IgA antibodies in secretions at sites distant from the gut, such as colostrums, lacrimal and salivary secretions in man and salivary secretions in rhesus monkeys and in rats.

A general conclusion therefore is that the secretory immune system can be stimulated centrally and that precursors of IgA-producing cells migrate from the gut-associated lymphoid tissue to several secretory sites in addition to the lamina propria of the gut itself. Therefore, if antigens are injected into the submucosal tissues, they are likely to induce serum IgG antibodies as well as secretory IgA antibodies in saliva. However, if it is applied topically to the skin or to the intraepiethelial tissue, secretory IgA is the main product which is detected in saliva. The role of topically applied antigen in the localization and persistence of IgA responses has been demonstrated in several secretory sites, including the respiratory tract, oral cavity, gut, and vagina.

The evidence that cells migrate from the gut to various secretory tissues, and that immunization in the gut leads to antibodies at various secretory sites has led to the concept of a common mucosal system. However, this concept may be an oversimplification, since although immunization in the lung may lead to antibodies in distant secretory sites, such as salivary glands and immunization in the lacrimal glands has also been shown to lead to the production of antibodies in saliva. Thus, with firm evidence that antigen deposition in the gut may lead to antibodies not only in the gut but also in saliva, lungs, lacrimal secretions and genitourinary tract, it is probably more correct to designate the system as an enteromucosal system.

Saliva is a source of body fluid for detection of an immune response to bacterial, food, and other antigens present in the oral cavity and gastrointestinal tract. Indeed, salivary antibody induction has been widely used as a model system to study secretory responses to ingested material, primarily because saliva is an easy secretion to collect and analyze. It seems to be a general feature that salivary IgA antibodies can be induced in a variety of species in the absence of serum antibodies. This has been demonstrated after immunization with particulate bacterial antigens in human could selectively induce an immune response to Streptococcus mutans by oral administration of the antigen. This route of administration resulted only in antibody production in saliva and not in serum. Similar mucosal immune response in the form of saliva IgA did occur in monkeys, rabbits, rats, and mice after oral administration of Streptococcus mutans or other bacteria.

This lack of production of IgG, but IgA production in saliva after oral or intragastric administration of bacterial antigens is shown in the following table.

TABLE 1
Induction of salivary IgA antibody after
stimulation of gut associated lymphoid tissue

| | | | Salivary | Serum |
|---|---|---|---|---|
| | | Route of | IgA | Antibody |
| Species | Antigen | Administration | Production | Production |
| Human | Streptococcus | Oral | ++ | − |
| | Mutans | | | |
| Monkeys | Streptococcus | Intragastric | ++ | − |
| | Mutans | | | |
| Rabbits | Penumococcus or | Intragastric | ++ | − |
| | BGG | | | |
| Rats | Streptococcus | Oral | ++ | − |
| | Mutans | | | |
| Mice | Streptococcus | Intragastric | ++ | − |
| | Mutans or | | | |
| | Ovalbumin | | | |

As indicated in this table, oral or intragastric administration of dietary soluble proteins such as bovine gammaglobulin (BGG) and ovalbumin or eggalbumin resulted in salivary IgA production but not in any antibody production in serum. For these reasons, saliva has been selected not only because of its relevance in oral disease, but mainly because it is an accessible fluid, easy to collect, and is thought to show representative responses in secretions after central or intragastric immunization. However, if both saliva IgA and serum IgG antibodies are detected in the same patient, it means that this individual has been primed with the antigen orally as well as systematically.

This IgA production in saliva and IgG production in serum is dependent upon antigen dosage as well as the integrity of the gut. For example, a single intragastric immunization with 1 mg of eggalbumin led to oral tolerance but did not lead to detectable secretory IgA antibodies, whereas 10 mg of ovalbumin led to systemic tolerance, but to a significant level of salivary IgA antibodies. Thus, detection of high levels of antibody in saliva is an indication of the body's exposure to significant levels of antigenic stimulation, such as 10 mg or higher.

While this concept of oral tolerance to high doses of soluble antigen may be correct, certain conditions—such as overloading of the GI tract with bacterial toxins—may not lead to oral tolerance. This is due to the fact that bacterial toxins will cause the opening of tight junctions, which will in turn lead to the absorption of ingested proteins and bacterial antigens from the gut in significant amounts. This excessive uptake of bacterial, fungal, viral, and dietary proteins into the circulation may induce immune response first in the form of IgM, and thereafter in the form of IgG and IgA antibodies in the serum, all of which may lead to different clinical conditions.

### SUMMARY OF THE INVENTION

One aspect of the preferred embodiment is a method for diagnosing the exposure of infectious agents in a patient. This method includes (a) determining a level of antibodies against an infectious agent or a corresponding recombinant antigen or synthetic peptide in a sample from the patient and (b) comparing the level of antibodies determined in step (a) with normal levels of the same antibodies.

Possible outcomes for the comparison include (i) normal levels of infectious antibodies indicate optimal conditions; (ii) higher than normal levels of infectious agent antibodies indicate a presence or possibility of an infection.

In one embodiment, the antigens are chosen from bacterial agent, parasitic agent, or viral agent.

In one embodiment, an ELISA test is used to determine the levels of antibodies.

In one embodiment, the antibodies, preferably IgA antibodies, are measured from saliva.

Further objects, features and other advantages of the preferred embodiments become apparent from the ensuing detailed description, considered together with the appended figures.

### BRIEF DESCRIPTION OF THE DRAWINGS

FIG. 1 is a graph showing saliva IgA antibodies against infectious agents, specific and non-specific autoantigens involved in cardiovascular disease and autoimmune disease expressed by O.D.'s from patients with possible cardiovascular disease.

FIG. 2 is a graph showing saliva IgA antibodies against infectious agents, specific and non-specific autoantigens involved in cardiovascular disease and autoimmune disease expressed by O.D.'s from patients with possible autoimmune disease.

FIG. 3 is a graph showing saliva IgA antibodies against infectious agents, specific and non-specific autoantigens involved in cardiovascular disease and autoimmune disease expressed by O.D.'s from healthy controls.

FIG. 4 is a graph showing the mean and standard deviation of thirty saliva samples of IgA antibody levels against oral bacteria, Chlamydia pneunoniae, and Mycoplasma species.

FIG. 5 is a graph showing the mean and standard deviation of thirty saliva samples of IgA antibody levels against Helicobacter pylori and Herpes viruses.

FIG. 6 is a table showing the correlation of reactivity of saliva IgA antibody against infectious agents to medical condition, such as infection.

### DETAILED DESCRIPTION OF THE PREFERRED EMBODIMENT

The inventor has developed a single test that will accurately inform the physician of important clinical conditions required to diagnosing in patients the likelihood and severity of infection. The test utilizes a highly sensitive and accurate ELISA test method that measures saliva IgA specific antibody titers to the purified antigens or a corresponding recombinant antigen or synthetic peptide from infectious agents.

Such quantitative and comparative test results allow the physician to determine exposure to infectious agents in patients. The test thus helps the clinical investigator to evaluate and treat patients by using immunological responses as indications of infection.

The test involves determining the level of antibodies against an infectious agent or a corresponding recombinant antigen or synthetic peptide. The level of antibodies against an infectious agent is compared between test samples of a patient and normal controls. A higher than normal level of antibodies against infectious agents indicate a presence or possibility of infection.

The presence of aerobic and anaerobic bacteria, along with other microorganisms found in the oral cavity, gastrointestinal tract, and blood may cause infections. These infectious agents include Streptococcus sanguis, Streptococcus oralis, Peptostreptococcus anaerobius, Eubacterium alactolyticum, Bacteroides oralis, Porphyromonas gingivalis, Borellia burdorferi, Treponema pallidium, Mycoplasma pneumoniae, Mycoplasma genitalium, Mycoplasma fermentens, Mycoplasma oralis, Chlamydia pneumoniae, Chlamydia trachomatis, Helicobacter pylori, Coxsackievirus, Epstein-Barr virus, cytomegalovirus, Hepatitis A, Trypanozoma cruzei, and other bacteria, parasites, or viruses.

Infection with these bacteria, parasites, or viruses results in a significant level of antibodies against different antigens, peptides, or epitopes of the infectious agents. The antibodies are present as saliva IgA.

Secretory IgA is capable of functioning as a blocking antibody, which can create a barrier to certain macromolecules, bacteria, and viruses. The interaction with secretory IgA will not permit such antigens to interact with the mucosa and blocks their entrance and exposure to the gut-associated lymphoid tissue. This blockage permits the host to shield efficiently the systemic immune response, local immune response, or both, from being bombarded by many molecules.

The properties of human IgA in serum and saliva are different. Serum IgA is monomeric and contains 80-90% IgA₁, and 10-20% IgA₂, while secretory IgA is polymeric and contains 50-75% IgA₁ and 25-50% IgA₂.

Because of these properties, secretory IgA can bind to the invading orgainisms more effectively. Therefore, secretory IgA have anti-bacterial, anti-fungal, and anti-viral activities, and play an important role in protection of mucosal surfaces from adherence of microorganisms. This prevention of colonization of the mucous membrane by secretory IgA is done by binding and blocking of specific binding sites on the bacterial cell wall.

Although other materials and methods similar or equivalent to those described herein can be used in the practice or testing of the preferred embodiments, the preferred method and materials are now described.

### EXAMPLE 1

### General Procedures for Infections Panel

For the test, about 2 ml of patient saliva was collected. Saliva specimen was kept at −20° C. until the performance of the assays.

The purified antigens were immobilized by attachment to a solid surface, such as a microtiter plate. The saliva sample was added to the plate followed by incubation and washing. Antibody bound to antigen was revealed by adding enzyme labeled monoclonal antibody directed against the first immunoglobulin. After addition of substrate, color development was measured by microtiter reader at 405 nm. The intensity of the color was directly related to the concentration of antibodies to these antigens present in patient's specimen.

Saliva samples were collected in the morning, before brushing teeth, smoking, or drinking. 2 ml of saliva was collected. Saliva was collected after a gentle chewing action in a test tube containing 0.1 ml of preservative. Saliva specimen was kept at −20° C. until the performance of the assays.

Calibrator samples I, II, III as well as positive and negative controls were used.

The wash buffer was made as follows: in a 500 ml graduated cylinder, 450 ml of water was added to 50 ml of 10× wash buffer. It was mixed and transferred to a 500 ml squeeze bottle and stored at 2-8° C. until used.

Substrate buffer and Stop Solution were ready for use. (CAUTION: Both solutions are caustic: avoid contact with skin and eyes, rinse with copious amounts of water in event of contact.)

The substrate solution was prepared only immediately before use. For 1-5 strips, 5 ml of substrate buffer were pipeted into the empty substrate reconstitution bottle and 1 substrate tablet was dropped in. The bottle was shaken to dissolve the tablet. The buffer was used within an hour after reconstitution as recommended.

Reagent and specimen were prepared as follows. All strips to be used, reagents, controls, and patient's specimen were equilibrated to room temperature (22-25° C.). Patient's specimen was diluted 1:100 with specimen diluent buffer: 20 μl specimen+2.0 ml buffer. Specimen dilutions were made in tubes prior to addition to wells and thoroughly mixed before dispensing. Only one well per test was necessary. For every determination, six strips (1-6) of eight wells were needed to run blank calibrators and four patient's samples.

Well Identification: 6 antigen-coated strips were used. Each was divided into 8 equal-sized squares. The top 6 squares were labeled “BLANK”, the next 3 were “CALIBRATOR I, CALIBRATOR II, and CALIBRATOR III”. The last 4 were labeled “SPECIMEN I, SPECIMEN II, SPECIMEN III and SPECIMEN IV”. Note: Blank and calibrators may need to be positioned differently if specified by the instrument manufacturer. For each test performance the following wells were used: One blank well (reagent blank), one well each for Calibrator I, II and III, and one well each for patient specimens.

The assay procedure was as follows: 100 μl of specimen diluent buffer was pipeted into all eight wells of strip #1, 2, 3, 4, 5, and 6. The contents were discarded and the addition of specimen diluent buffer to the same wells was repeated. Then, 100 μl of each calibrator or patient specimen dilutions were pipetted into identified wells; being careful to avoid splashing and air bubbles because cross-contamination between the wells may cause erroneous results. Then, 100 μl of specimen diluent buffer was pipeted into a blank well. The reagents were dispensed slowly to avoid splashing and air bubbles. If large air bubbles occurred, they were aspirated or the plate was gently shaken. The plate was covered and incubated for 60 minutes at room temperature (22-25° C.). Specimen was shaken from the wells into a container containing disinfectant solution or aspirated with a vacuum device. All wells were empty prior to filling with 1×wash buffer and allowing a 10-20 second soak time. The wells were emptied by shaking into a disposal container or aspirated. Washing was repeated three more times. The inverted plate was tapped onto a paper towel to completely remove all residual liquid. Then, 100 μl of anti IgA conjugate was added to the tested strips. The plate was covered and incubated for 60 minutes at room temperature (22-25° C.). The liquid was shaken or aspirated from all the wells and washed four times. Then, 100 μl of p-NPP substrate was added to all the wells at timed intervals that corresponded to the reading time of the instrument used to read the reactions. The 45-minute incubation time was started as substrate was added to the first well. The plate was covered and incubated 45 minutes at 22-25° C. (The assay may be incubated for less than 45 minutes if incubation temperature is higher than 25° C.). Then, 50 μl of 3N NaOH was pipeted into all the wells at the same timed intervals that the p-NPP was added. The plate was shaken for 1-2 minutes by hand or on a shaker, avoiding splashing. The bottom of the wells was wiped with a non-abrasive paper towel and the instrument was zeroed on the blank well. The OD was read at 405±5 nm within 30-minutes, and reactions recorded.

The ELISA values for the calibrators used in this test system were according to the samples used in the test.

ELISA values for each test specimen were determined using the following formula:

$$\begin{matrix}ELISA values \\ of test \\ specimen\end{matrix} = \frac{Values of calibrator \times Absorbance of test specimen}{Absorbance of calibrator }$$

### EXAMPLE 2

### Test for Bacterial Specific Antibodies

Different strains of oral bacteria, including Streptococcus Sanguis, Streptococcus Oralis, Peptostreptococcus Anaerobius, Eubacterium Alactolyticum, Bacteroides Oralis, Porphyromonas Gingivalis, and others, were purchased from American Type Culture Collection in Rockville, Md. The bacteria were lysed by sonication and the purified antigens were immobilized by attachment to a solid surface, such as a microtiter plate. The test specimen was added to the plate followed by incubation and washing. The procedure in Example 1 was followed to measure for the bacterial specific antibodies.

The ELISA values for the calibrators used in this test system were as follows: Calibrator I=20, Calibrator II=50, and Calibrator III=200.

The ELISA values for each test specimen were determined using the formula in Example 1.

### EXAMPLE 3

### Test for Bacterial and Viral Specific Antibodies

The experiments are limited to microorganisms both possessing the MA'ST motif and implicated in heart disease. Borrelia burgdorferi, the spirochete causing Lyme disease; Treponema pallidum, the causative agent of syphilis; Mycoplasma pneumoniae, an etiologic agent of non-viral primary atypical pneumonia; Mycoplasma genitalium, associated with urogenital infection; and Heliocobacter pylori, associated with duodenal and gastric ulcers; as well as the protozoan Trypanosoma cruzi, the cause of Chagas disease.

The following peptides were synthesized by a robotic multiple peptide synthesizer:

| Infectious agent | | Peptide portion |
|---|---|---|
| 1. | Chlamydia pneumoniae | LPTAVLNLTAWNPSLLGNATALST |
| 2. | Mycoplasma pneumoniae | TPPNMATLVSTAMSL |
| 3. | Borrelia burgdorferi | LFLIMATFLSPSISG |
| 4. | Treponema pallidum | RSEAMALVLSTLENR |
| 5. | Trypanozoma cruzi | NTFHMAGGGSTLINL |
| 6. | Coxsackie virus | FIEWLKVKILPEVKEKHEFLSRL |
| 7. | Epstein-Barr virus | TPSPAIPSHSSNTALERPLA |
| 8. | Cytomegalovirus | VMAPRTLILTVGLLCMRI |
| 9. | Helicobacter pylori | TYNBMATGTSPVMSG |
| 10. | Streptococcus Group-A | RVTTRSQAQDAAGLKEKADC |

Peptides were characterized by reversed-phased HPLC and electrospray mass-spectrometry with purity greater than 80%. These peptides were bound to bovine serum albumin and used for coating microtiter plates. The test specimen was added to the plate followed by incubation and washing. The procedure in Example 1 was followed to measure for the bacterial and viral specific antibodies.

The ELISA values for the calibrators used in this test system were as follows: Calibrator I=50, Calibrator II=100, and Calibrator III=400.

The ELISA values for each test specimen were determined using the formula in Example 1.

### EXAMPLE 4

### Analysis of Results

The results are analyzed as a panel. The values for bacteria, viruses, and parasites were obtained from a set of healthy controls.

Thirty patients (15 men and 15 women) with known risk factors for cardiovascular disease were tested. These patients have a blood pressure greater than 140/80 and cholesterol/HDL level greater than 7.

Thirty patients (15 men and 15 women) with known risk factors for autoimmune disease were tested. These patients have a positive ANA titer of 1:160 or greater and a rheumatoid factor of 50 international units or higher.

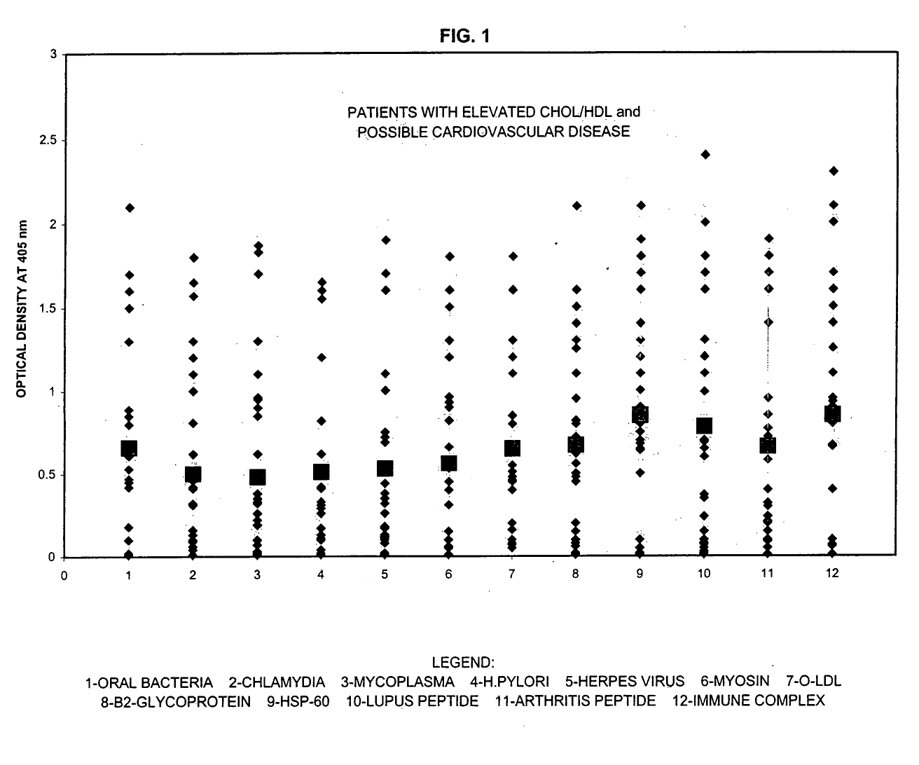

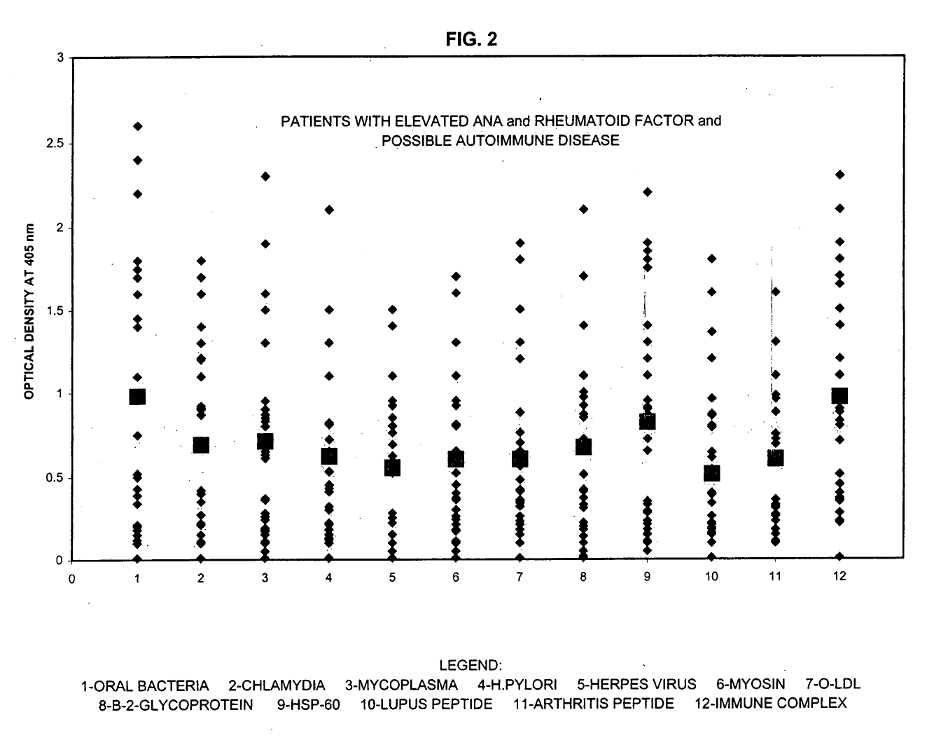

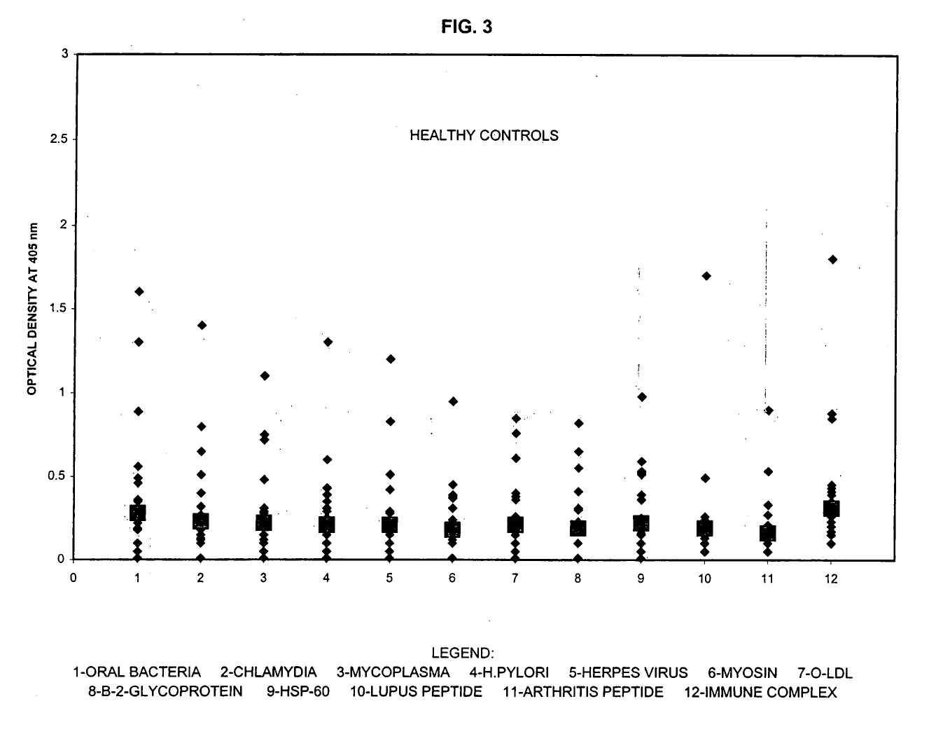

The assays for antibodies were performed according to the preceding Examples. The results summarized in FIGS. 1-5 are expressed based on optical densities, which are easily converted to ELISA units.

Tables 2-4 and FIGS. 1-5 summarizes the saliva IgA antibody levels against infectious agents in patients with possible cardiovascular disease, autoimmune disease and healthy control subjects.

TABLE 2
Saliva IgA Antibodies Against Infectious Agents Involved in Cardiovascular and
Autoimmune Disease Expressed by O.D.'s From Patients With Possible Cardiovascular
Disease

| | ORAL | | | | HERPES |
|---|---|---|---|---|---|
| SUBJECTS | BACTERIA | CHLAMYDIA | MYCOPLASMA | H. PYLORI | VIRUS |
| 1 | 0.45 | 0.32 | 0.22 | 0.31 | 0.26 |
| 2 | 0.89 | 0.41 | 0.33 | 0.29 | 0.32 |
| 3 | 0.1 | 0.06 | 0.01 | 0.04 | 0.13 |
| 4 | 0.02 | 0.01 | 0.07 | 0.02 | 0.01 |
| 5 | 0.42 | 0.09 | 0.02 | 0.17 | 0.12 |
| 6 | 1.5 | 1 | 1.7 | 1.55 | 1 |
| 7 | 0.01 | 0.01 | 0.03 | 0.02 | 0.08 |
| 8 | 2.1 | 1.1 | 0.96 | 1.2 | 1.1 |
| 9 | 1.6 | 1.57 | 1.87 | 1.6 | 1.7 |
| 10 | 1.3 | 1.65 | 1.83 | 1.65 | 1.6 |
| 11 | 0.18 | 0.13 | 0.1 | 0.13 | 0.17 |
| 12 | 0.47 | 0.13 | 0.19 | 0.13 | 0.18 |
| 13 | 0.1 | 0.1 | 0.1 | 0.11 | 0.11 |
| 14 | 0.1 | 0.01 | 0.01 | 0.01 | 0.01 |
| 15 | 0.53 | 0.16 | 0.38 | 0.31 | 0.35 |
| 16 | 0.42 | 0.46 | 0.01 | 0.01 | 0.08 |
| 17 | 0.1 | 0.04 | 0.02 | 0.01 | 0.1 |
| 18 | 0.1 | 0.01 | 0.03 | 0.04 | 0.01 |
| 19 | 0.02 | 0.01 | 0.01 | 0.02 | 0.02 |
| 20 | 0.02 | 0.06 | 0.01 | 0.17 | 0.1 |
| 21 | 1.3 | 1.8 | 1.1 | 1.55 | 1.9 |
| 22 | 1.6 | 1.3 | 0.9 | 0.82 | 0.75 |
| 23 | 0.8 | 0.62 | 0.62 | 0.41 | 0.38 |
| 24 | 0.42 | 0.81 | 0.95 | 0.62 | 0.69 |
| 25 | 0.61 | 0.31 | 0.35 | 0.26 | 0.51 |
| 26 | 1.5 | 1.2 | 1.3 | 1.6 | 1.1 |
| 27 | 0.85 | 0.42 | 0.32 | 0.52 | 0.72 |
| 28 | 0.1 | 0.1 | 0.01 | 0.1 | 0.1 |
| 29 | 1.7 | 1.1 | 0.85 | 0.42 | 1.6 |
| 30 | 0.65 | 0.32 | 0.26 | 0.33 | 0.44 |
| Mean +/− S.D. | 0.66 +/− 0.61 | 0.50 +/− 0.56 | 0.48 +/− 0.58 | 0.51 +/− 0.55 | 0.53 +/− 0.56 |

TABLE 3
Saliva IgA Antibodies Against Infectious Agents Involved in Cardiovascular and
Autoimmune Disease Expressed by O.D.'s From Patients With Possible Autoimmune
Disease

| | ORAL | | | | HERPES |
|---|---|---|---|---|---|
| SUBJECTS | BACTERIA | CHLAMYDIA | MYCOPLASMA | H. PYLORI | VIRUS |
| 1 | 0.21 | 0.1 | 0.15 | 0.22 | 0.15 |
| 2 | 1.8 | 0.92 | 0.85 | 0.41 | 0.62 |
| 3 | 1.1 | 1.4 | 1.6 | 0.63 | 0.59 |
| 4 | 0.43 | 0.21 | 0.28 | 0.3 | 0.15 |
| 5 | 0.39 | 0.22 | 0.17 | 0.15 | 0.22 |
| 6 | 0.01 | 0.11 | 0.1 | 0.13 | 0.01 |
| 7 | 0.2 | 0.15 | 0.11 | 0.21 | 0.15 |
| 8 | 1.6 | 1.3 | 1.9 | 0.82 | 0.76 |
| 9 | 2.2 | 1.7 | 1.6 | 1.3 | 0.85 |
| 10 | D | 0.9 | 1.5 | 0.81 | 0.92 |
| 11 | 1.4 | 1.1 | 0.83 | 0.45 | 0.52 |
| 12 | 0.34 | 0.21 | 0.26 | 0.18 | 0.15 |
| 13 | 1.45 | 1.21 | 1.3 | 1.5 | 1.1 |
| 14 | 0.75 | 0.42 | 0.36 | 0.43 | 0.54 |
| 15 | 0.1 | 0.1 | 0.01 | 0.01 | 0.01 |
| 16 | 0.52 | 0.91 | 0.95 | 0.72 | 0.69 |
| 17 | 0.21 | 0.15 | 0.18 | 0.22 | 0.25 |
| 18 | 2.4 | 1.8 | 0.9 | 1.3 | 1.5 |
| 19 | 1.7 | 1.2 | 0.65 | 1.1 | 0.95 |
| 20 | 1.1 | 0.92 | 0.87 | 1.3 | 1.4 |
| 21 | 0.5 | 0.35 | 0.61 | 0.32 | 0.52 |
| 22 | 1.6 | 0.4 | 0.37 | 0.53 | 0.1 |
| 23 | 0.18 | 0.11 | 0.19 | 0.1 | 0.05 |
| 24 | 0.34 | 0.27 | 0.36 | 0.21 | 0.28 |
| 25 | 0.98 | 0.87 | 0.63 | 1.1 | 0.95 |
| 26 | 0.12 | 0.01 | 0.05 | 0.12 | 0.1 |
| 27 | 1.75 | 1.6 | 1.3 | 2.1 | 0.95 |
| 28 | 2.6 | 1.1 | 2.3 | 1.3 | 0.8 |
| 29 | 1.4 | 0.9 | 0.8 | 0.65 | 0.92 |
| 30 | 0.15 | 0.1 | 0.24 | 0.15 | 0.05 |
| Mean +/− S.D. | 0.98 +/− 0.76 | 0.69 +/− 0.55 | 0.71 +/− 0.60 | 0.62 +/− 0.52 | 0.55 +/− 0.42 |

TABLE 4
Saliva IgA Antibodies Against Infectious Agents Involved in Cardiovascular and
Autoimmune Disease Expressed by O.D.'s From Healthy Controls

| | ORAL | | | | |
|---|---|---|---|---|---|
| SUBJECTS | BACTERIA | CHLAMYDIA | MYCOPLASMA | H. PYLORI | HERPES VIRUS |
| 1 | 0.1 | 0.1 | 0.05 | 0.1 | 0.1 |
| 2 | 0.05 | 0.1 | 0.05 | 0.1 | 0.1 |
| 3 | 0.36 | 0.21 | 0.22 | 0.18 | 0.15 |
| 4 | 0.01 | 0.1 | 0.1 | 0.1 | 0.1 |
| 5 | 0.1 | 0.1 | 0.1 | 0.1 | 0.15 |
| 6 | 0.46 | 0.32 | 0.29 | 0.35 | 0.29 |
| 7 | 0.01 | 0.1 | 0.01 | 0.01 | 0.01 |
| 8 | 0.19 | 0.15 | 0.1 | 0.1 | 0.1 |
| 9 | 0.18 | 0.1 | 0.15 | 0.1 | 0.1 |
| 10 | 0.1 | 0.1 | 0.1 | 0.1 | 0.1 |
| 11 | 0.22 | 0.18 | 0.26 | 0.25 | 0.28 |
| 12 | 0.89 | 0.65 | 0.72 | 0.43 | 0.51 |
| 13 | 0.01 | 0.15 | 0.1 | 0.1 | 0.1 |
| 14 | 0.1 | 0.1 | 0.1 | 0.1 | 0.1 |
| 15 | 0.35 | 0.27 | 0.31 | 0.29 | 0.15 |
| 16 | 0.1 | 0.12 | 0.1 | 0.15 | 0.1 |
| 17 | 0.24 | 0.21 | 0.26 | 0.2 | 0.17 |
| 18 | 1.3 | 0.8 | 0.75 | 0.6 | 0.83 |
| 19 | 0.1 | 0.1 | 0.1 | 0.1 | 0.1 |
| 20 | 0.01 | 0.01 | 0.01 | 0.01 | 0.05 |
| 21 | 0.27 | 0.21 | 0.25 | 0.19 | 0.15 |
| 22 | 0.1 | 0.1 | 0.1 | 0.1 | 0.1 |
| 23 | 0.18 | 0.15 | 0.21 | 0.2 | 0.29 |
| 24 | 1.6 | 1.4 | 1.1 | 1.3 | 1.2 |
| 25 | 0.25 | 0.13 | 0.12 | 0.1 | 0.1 |
| 26 | 0.05 | 0.1 | 0.01 | 0.1 | 0.1 |
| 27 | 0.49 | 0.4 | 0.28 | 0.31 | 0.28 |
| 28 | 0.1 | 0.1 | 0.1 | 0.1 | 0.1 |
| 29 | 0.56 | 0.51 | 0.48 | 0.39 | 0.42 |
| 30 | 0.1 | 0.01 | 0.01 | 0.05 | 0.2 |
| Mean +/− S.D. | 0.28 +/− 0.37 | 0.23 +/− 0.28 | 0.22 +/− 0.24 | 0.21 +/− 0.24 | 0.21 +/− 0.23 |

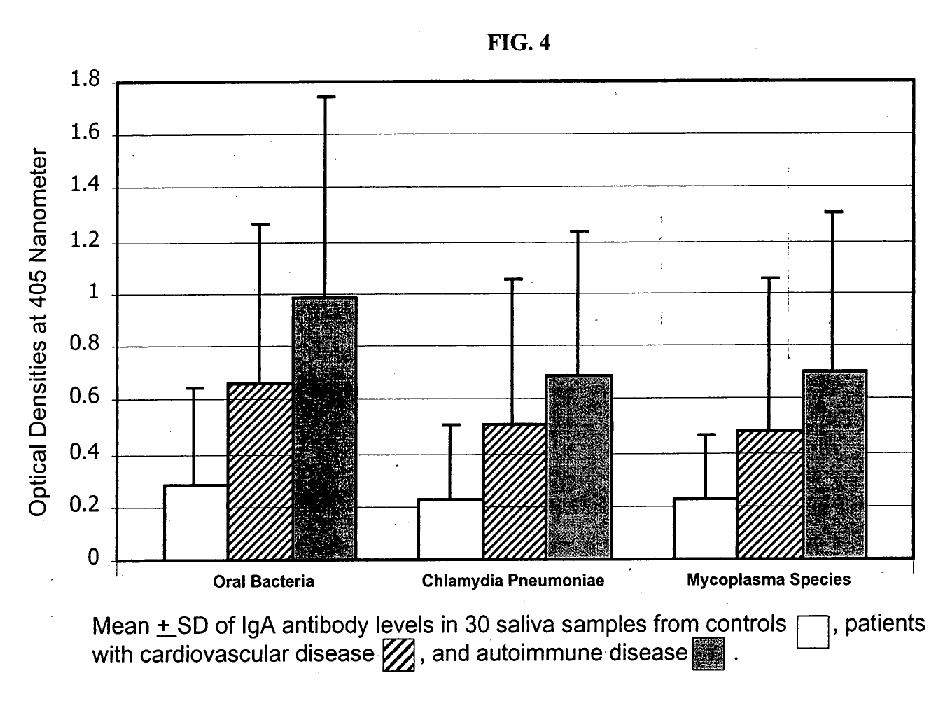

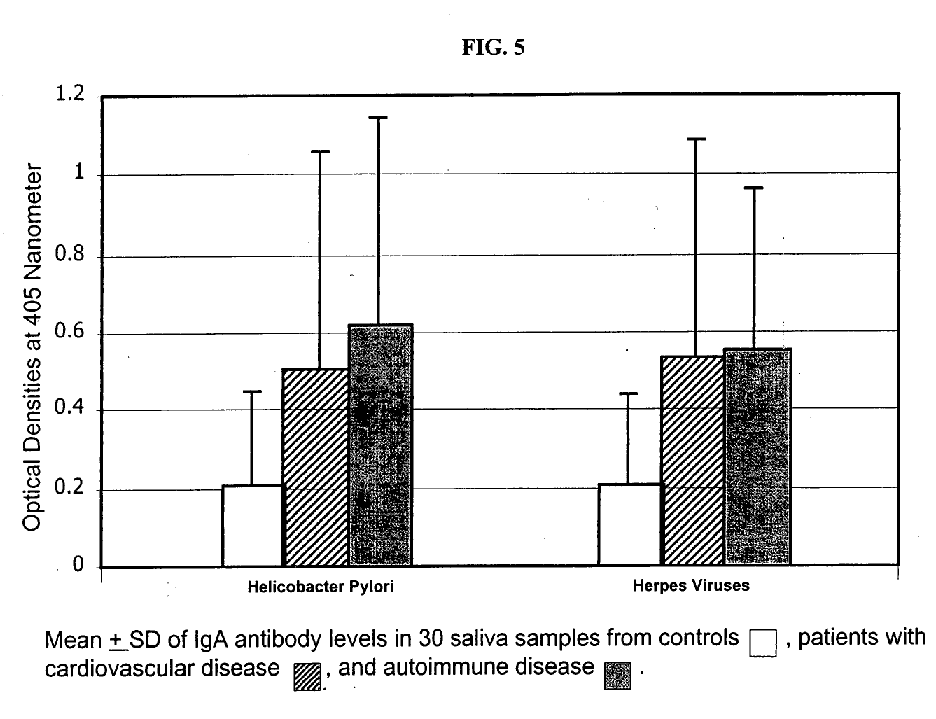

FIGS. 1-3 illustrate each optical density as well as the mean of saliva IgA antibody level against 12 antigens. FIGS. 4 and 5 illustrate the mean and standard deviation of saliva IgA antibody levels from healthy controls, patients with cardiovascular disease, and patients with autoimmune disease.

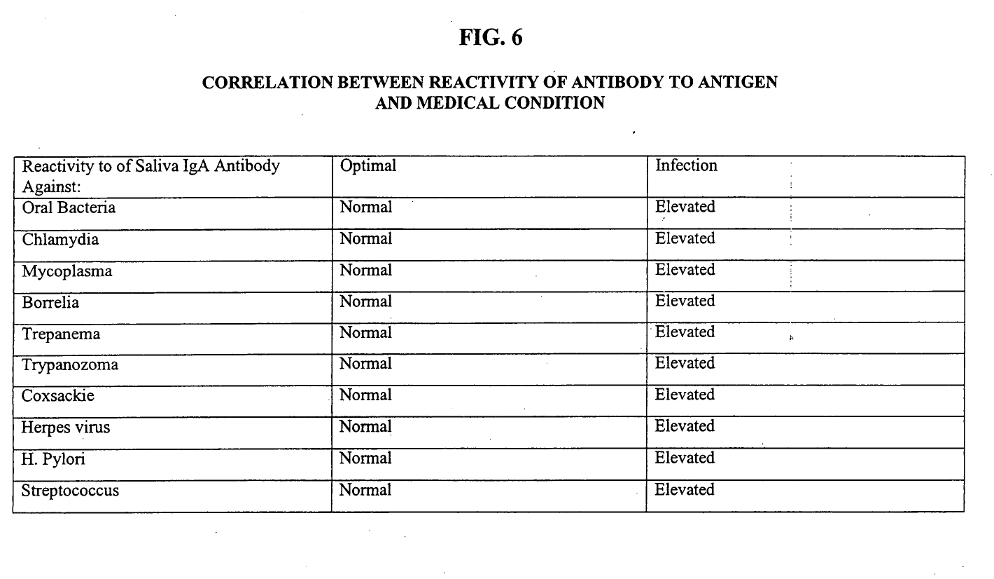

FIG. 6 shows data interpretation of antibody levels to infectious agents relating to the possibility or presence of infection. The detection of above normal levels of saliva IgA antibody against the antigens listed in FIG. 6 can help diagnose a possibility or presence of infection. A normal level of antibody is defined as an average level of antibody taken from a set of healthy control individuals. For instance, the average levels are shown as the big squares on FIGS. 1-3.

Absence of secretory IgA is the most immunodeficiency disorder, accounting for 15% of all primary immunodeficiency syndrome cases. Frequency of certain diseases, mainly neurological (24%), gastrointestinal 928%), collagen and autoimmune (20%), and recurrent infections (23%) may occur in patients with selective IgA deficiency. These include neuropathies, endocrinopathies, atopy, celiac disease, asthma, food allergies, rheumatoid arthritis, lupus, malabsorption syndrome, lymphomas, bacterial, viral, and fungal infections. High levels of total secretory IgA may indicate viral infection or overgrowth of yeast aerobic and anaerobic bacteria in the oral cavity as well as in different parts of the gastrointestinal tract. These infectious agents may directly or indirectly contribute to the occurrence of cardiovascular and autoimmune diseases.

The results of the test panels shown in combination with other clinical data and evaluation by the clinician allows for a faster and more accurate diagnosis of the above indications.

1. A method for diagnosing a possibility of cardiovascular disease or autoimmune disease in a patient, comprising the steps of:
a) obtaining a sample from said patient
b) determining a level of antibodies in the sample, said antibodies being directed against an infectious agent associated with pathogenesis of cardiovascular disease or autoimmune disease or a corresponding recombinant antigen or synthetic peptide;
c) comparing the level of antibodies determined in step b) with normal levels of said antibodies, wherein
normal levels of infectious antibodies indicate optimal conditions; and
higher than normal levels of infectious agent antibodies indicate the possibility of cardiovascular disease or autoimmune disease.

2. The method according to claim 1, wherein step a) comprises determining a level of antibodies against an infectious agent selected from the group consisting of bacterial agent, parasitic agent, and viral agent.

3. The method according to claim 1, wherein step a) comprises determining a level of antibodies against an infectious agent selected from the group consisting of Streptococcus sanguis, Streptococcus oralis, Peptostreptococcus anaerobius, Eubacterium alactolyticum, Bacteroides oralis, Porphyromonas gingivalis, Borellia burdorferi, Treponema pallidium, Mycoplasma pneumoniae, Mycoplasma genitalium, Mycoplasma fermentens, Mycoplasma oralis, Chlamydia pneumoniae, Chlamydia trachomatis, Helicobacter pylori, Coxsackievirus, Epstein-Barr virus, Cytomegalovirus, Hepatitis A, and Trypanozoma cruzei.

4. The method according to claim 1, wherein determining the level of antibodies in steps a) and b) is accomplished using an immunoassay.

5. The method according to claim 6, wherein the immunoassay is an ELISA test.

6. The method according to claim 1, wherein the antibodies in steps a) and b) is measured from saliva.

7. The method according to claim 6, wherein the measured antibodies are IgA.


SEQUENCE LISTING (11 sequences)

SEQ ID NO: 1 - protein, 15 aa, mouse M7A-alpha peptide MA′ST motif
SLKLMATLFSTYASA

SEQ ID NO: 2 - protein, 24 aa, Chlamydia pneumoniae peptide
LPTAVLNLTAWNPSLLGNATALST

SEQ ID NO: 3 - protein, 15 aa, Mycoplasma pneumoniae peptide
TPPNMATLVSTAMSL

SEQ ID NO: 4 - protein, 15 aa, Borrelia burgdorferi peptide
LFLIMATFLSPSISG

SEQ ID NO: 5 - protein, 15 aa, Treponema pallidum
RSEAMALVLSTLENR

SEQ ID NO: 6 - protein, 15 aa, Trypanozoma cruzi peptide
NTFHMAGGGSTLINL

SEQ ID NO: 7 - protein, 23 aa, Coxsackie virus peptide
FIEWLKVKILPEVKEKHEFLSRL

SEQ ID NO: 8 - protein, 20 aa, Epstein-Barr virus peptide
TPSPAIPSHSSNTALERPLA

SEQ ID NO: 9 - protein, 18 aa, Cytomegalovirus peptide
VMAPRTLILTVGLLCMRI

SEQ ID NO: 10 - protein, 15 aa, Helicobacter pylori peptide
TYNBMATGTSPVMSG

SEQ ID NO: 11 - protein, 20 aa, Streptococcus Group-A peptide
RVTTRSQAQDAAGLKEKADC